(19) United States
(12) Patent Application Publication
(10) Pub. No.: US 2019/0151492 A1
(43) Pub. Date: May 23, 2019

(54) CATALYST DEVICE AND AIR CONDITIONING APPARATUS FOR VEHICLE HAVING THE SAME

(71) Applicant: Hanon Systems, Daejeon (KR)

(72) Inventors: Jae Ho KIM, Daejeon (KR); Ki Hong KIM, Daejeon (KR); Ji Yong PARK, Daejeon (KR); Ho Chang SIM, Daejeon (KR); Yong Jun JEE, Daejeon (KR); Jae Woo KO, Daejeon (KR); Tae Yong PARK, Daejeon (KR)

(21) Appl. No.: 16/096,328

(22) PCT Filed: Aug. 10, 2017
(86) PCT No.: PCT/KR2017/008691

§ 371 Date: Oct. 25, 2018

(30) Foreign Application Priority Data
Aug. 12, 2016 (KR) 10-2016-0103111
Jul. 26, 2017 (KR) 10-2017-0094778

(51) Int. Cl. A61L 9/20; B60H 3/00

(52) U.S. Cl. CPC A61L 9/205; A61L 2209/12; B60H 3/0092

(57) ABSTRACT

The present invention relates to a catalytic device and a vehicle air conditioning apparatus comprising the same and, more particularly, provides a catalytic device comprising: a case (140); a light source part (120) disposed to face an inner side of the case (140) so as to radiate light toward an inner surface of the case (140); and a catalyst part (130) which is disposed on the inner surface of the case (140) and in which a photocatalytic reaction is induced by light radiated from the light source part (120), wherein the catalyst part (130) is located at a first separation distance (L) from the light source part (120) in order that a maximum light energy (Pmax) of the light source part (120) is focused thereon.

### TECHNICAL FIELD

The present disclosure relates to a catalyst device and an air conditioning apparatus for a vehicle having the same, and more particularly, to a catalyst device and an air conditioning apparatus for a vehicle having the same, which can adjust the optimum distance and angle between a light source part and a catalyst part in order to purify the air flowed into an air conditioning case, stably sterilize an evaporator, and safely protect the passenger in a vehicle, thus implementing the maximum performance of sterilization and deodorization for the evaporator.

### BACKGROUND ART

An air conditioning apparatus for a vehicle, as an apparatus for cooling or heating inside the vehicle by heating or cooling the air in the process of flowing the external air of the vehicle into the vehicle or circulating the air of the vehicle interior, includes an evaporator for cooling operation and a heater core for heating operation inside an air conditioning case and uses a blowing mode switching door to selectively blow the air cooled or heated by the evaporator or the heater core into each part in the vehicle interior.

Japanese Patent No. 2549032 (registered on May 30, 1997, entitled “Cooling apparatus with deodorizer for vehicle”), which has been earlier filed, has disclosed a cooling apparatus with a deodorizer for a vehicle.

FIG. 1 is a cross-sectional diagram illustrating the conventional cooling apparatus with the deodorizer for the vehicle.

Referring to FIG. 1, the conventional cooling apparatus with the deodorizer for the vehicle is provided with an external air suction port 21 and an internal air suction port 22 in a case 20, and pivotally with an intake door 23 that selectively opens and closes the external air suction port 21 and the internal air suction port 22. An actuator 30 is connected to the pivot shaft of the intake door 23 and is controlled by a control means 31.

The intake door 23 is provided at the downstream side thereof with a blower 25 for blowing the air flowed from the internal and external air suction ports 21, 22 toward the downstream side thereof, and the blower 25 is composed of a fan 32 and a motor 33 rotating the fan 32. An evaporator 26 is located at the downstream side of the blower 25 to exchange heat with the air passing through it, thus cooling the air.

An air passage 28 formed at the downstream side of the evaporator 26 is provided with a catalyst filter 27 for generating active oxygen by irradiating the light having a long wavelength.

The catalyst filter 27 generates active oxygen by the irradiation of an ultraviolet lamp 29, and the active oxygen oxidizes and decomposes the substance causing the odor to a very low concentration oxidizable compound. The ultraviolet lamp 29 is interposed between the evaporator 26 and the catalyst filter 27.

A metal catalyst filter 34 for removing ozone contained in the flowing air is provided at the downstream side of the catalyst filter 27. Reference numeral 35 refers to a temperature sensor, 36 to a sensor for sensing the odor level, 37 to a fan switch, and 24 to an air discharge port, which are not described.

However, there is a problem in that in the conventional cooling apparatus with the deodorizer for the vehicle, the ultraviolet lamp 29 used as the light source of the catalyst contains mercury that is harmful to the human body therein, and cannot be applied to the vehicle due to various environmental requirements.

In addition, there is a problem in that the catalyst filter 27 is provided at the downstream side of the evaporator 26 to absorb and deodorize the odor generated in the evaporator 26, such that the filter needs to be replaced due to a decrease in the air amount caused by an excessive amount of dust. In addition, there is a problem in that in the conventional cooling apparatus with the deodorizer for the vehicle, the ultraviolet lamp 29 and the catalyst filter 27 are separate parts, thus reducing the assemblability.

### DISCLOSURE

### Technical Problem

The present disclosure is intended to solve the above problems, and according to an embodiment of the present disclosure, an object of the present disclosure is to provide a catalyst device and an air conditioning apparatus for a vehicle having the same, which can adjust the spacing distance and angle between the light source part and the catalyst part at a specific distance and a specific angle to purify the air flowed into an air conditioning case and sterilize and deodorize the evaporator to concentrate the optical energy on the catalyst part in the optimal state, thus performing the sterilization and deodorization for the evaporator.

### Technical Solution

A catalyst device in accordance with an embodiment of the present disclosure includes a case 140, a light source part 120 located to face the inside of the case 140 to irradiate light toward the inside surface of the case 140, and a catalyst part 130 located on the inside surface of the case 140, and generating the photocatalyst reaction by the light irradiated by the light source part 120; and the catalyst part 130 is located to be spaced at a first spacing distance L apart from the light source part 120 so that the maximum optical energy Pmax of the light source part 120 is concentrated thereon.

The light source part 120 selectively uses any one of ultraviolet ray having a first wavelength region or visible ray having a second wavelength region.

The first spacing distance L is kept in the state spaced at the distance corresponding to ⅔*P intensity covering only the lower surface region of the catalyst part 130 based on the vertical distance between the light source part 120 and the catalyst part 130 when the optical amount P is irradiated from the light source part 120.

The first spacing distance L is kept at the length of the catalyst part*½*tan θ/2.

The first spacing distance L with the catalyst part 130 based on the light source part 120 is kept at 15 mm.

The diffusion angle (θ) of the light source part 120 based on the maximum optical energy of the light source part 120 is in a range of 20 degrees to 60 degrees.

The light source part 120 uses an LED.

The light source part 120 is composed of any one of one or in plural.

The catalyst part 130 is extended with the same horizontal and vertical lengths.

The first wavelength region has the optical amount P kept in a range of 180 nm to 380 nm.

The second wavelength region has the optical amount P kept in a range of 380 nm to 760 nm.

The second wavelength region has the optical amount P kept in a range of 400 nm to 500 nm.

The catalyst device includes a reflection plate located to face the light source part 120 on the inside of the case 140 to reflect the light source irradiated from the light source part 120 to the catalyst part 130.

The reflection plate 160 is located at the location between ⅔*P and ⅓*P based on the optical amount P.

The catalyst part 130 has the air porosity kept at 80% or more.

A catalyst device and an air conditioning apparatus for a vehicle having the same in accordance with another embodiment of the present disclosure includes an air conditioning case 300 for forming a space where inflow air is transferred to form a vent 310 through which the air is discharged; an evaporator 410 provided inside the air conditioning case 300; a heater core 420 provided at the rear side of the air conditioning case 300 in the air flow direction; and a catalyst device 100 of any one of claims 1 to 14.

The catalyst device 100 is provided at the front side of the evaporator 410 in the air flow direction.

The catalyst device 100 is provided at the rear side of the evaporator 410 in the air flow direction.

### Advantageous Effects

The catalyst device and the air conditioning apparatus for the vehicle having the same in accordance with an embodiment of the present disclosure can sterilize and deodorize the evaporator using the LED or visible light so that the passengers in the vehicle can avoid the problem caused by heavy metal poisoning.

The catalyst device and the air conditioning apparatus for the vehicle having the same in accordance with an embodiment of the present disclosure can locate the catalyst part on the location where the optical energy irradiated from the light source part is maximized, thus enhancing the deodorization effect of the catalyst part.

The catalyst device and the air conditioning device for the vehicle having the same in accordance with the embodiment of the present disclosure minimizes the size of the catalyst part and enhances the deodorization effect, thus achieving the stable sterilization regardless of the change of the catalyst amount.

### DESCRIPTION OF DRAWINGS

FIG. 1 is a cross-sectional diagram illustrating the conventional cooling apparatus with the deodorizer for the vehicle.

FIG. 2 is an exploded perspective diagram illustrating a catalyst device in accordance with a first embodiment of the present disclosure.

FIG. 3 is a longitudinal cross-sectional diagram of the state in which FIG. 2 has been assembled.

FIG. 4 is a diagram illustrating the spacing distance between the conventional light source part and catalyst part.

FIG. 5 is a diagram illustrating the spacing distance between a light source part and a catalyst part in accordance with an embodiment of the present disclosure.

FIG. 6 is a diagram illustrating a reflection plate provided in the catalyst device in accordance with the embodiment of the present disclosure.

FIG. 7 is a graph illustrating the deodorization state depending upon the elapsed time through the catalyst device in accordance with an embodiment of the present disclosure.

FIG. 8 is a diagram illustrating an air conditioning apparatus for a vehicle in accordance with another embodiment of the present disclosure.

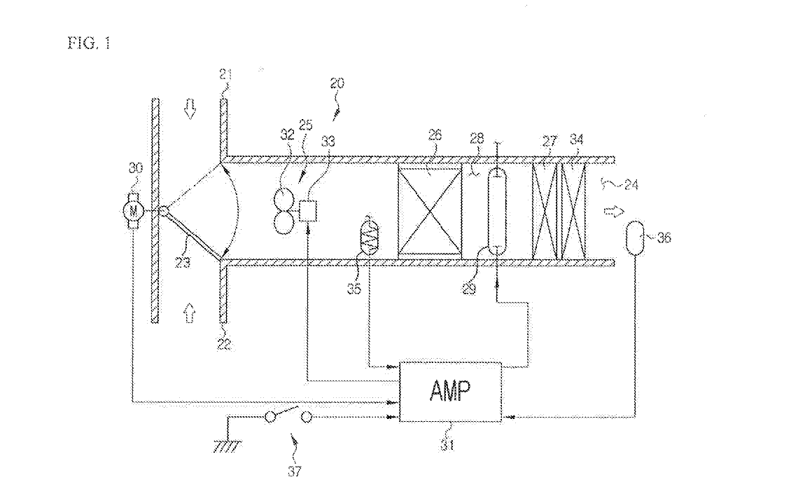

### BEST MODE

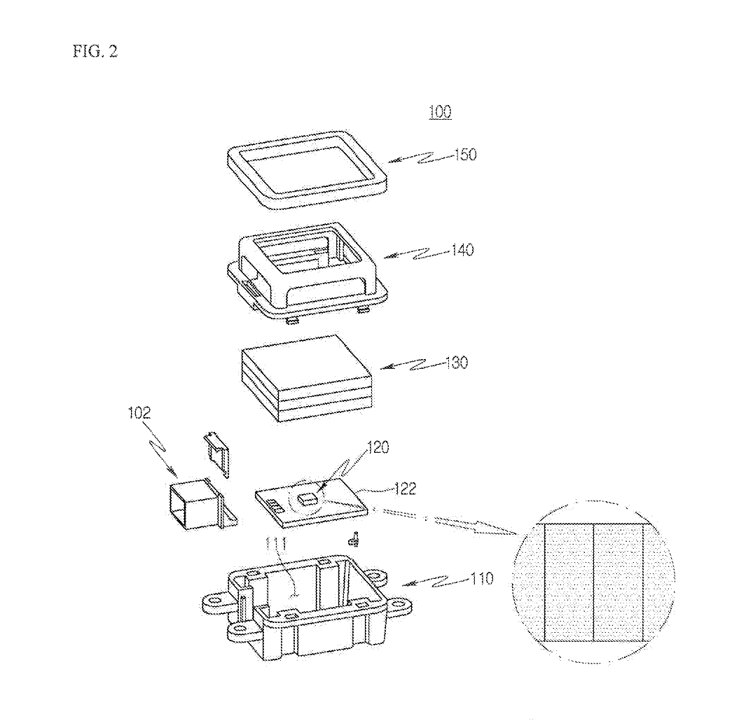

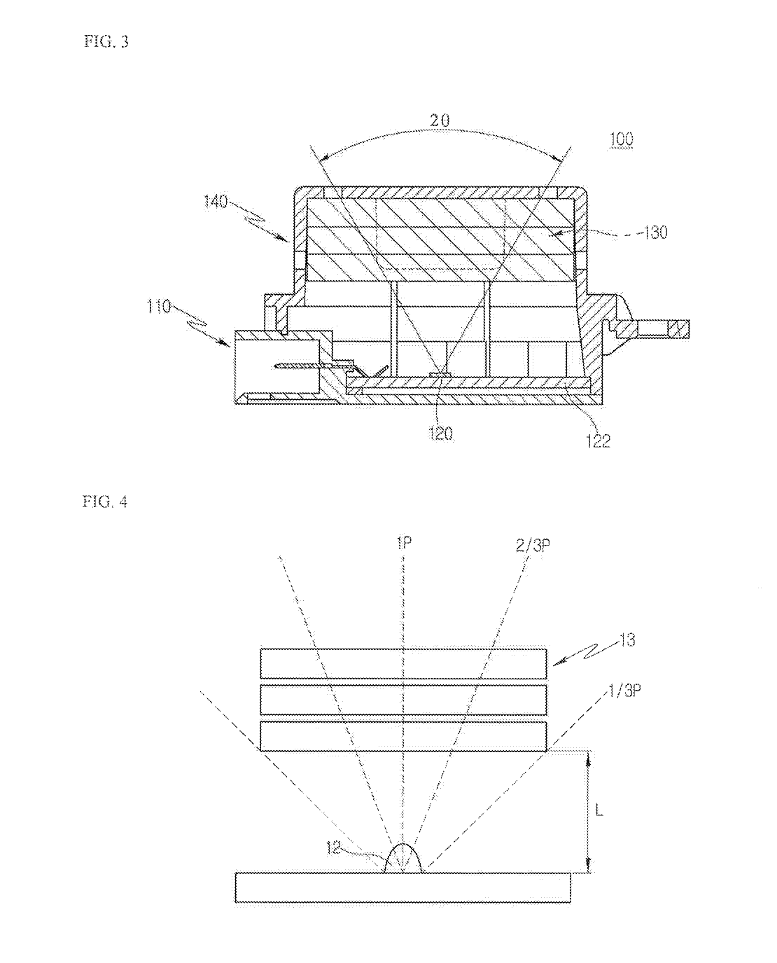

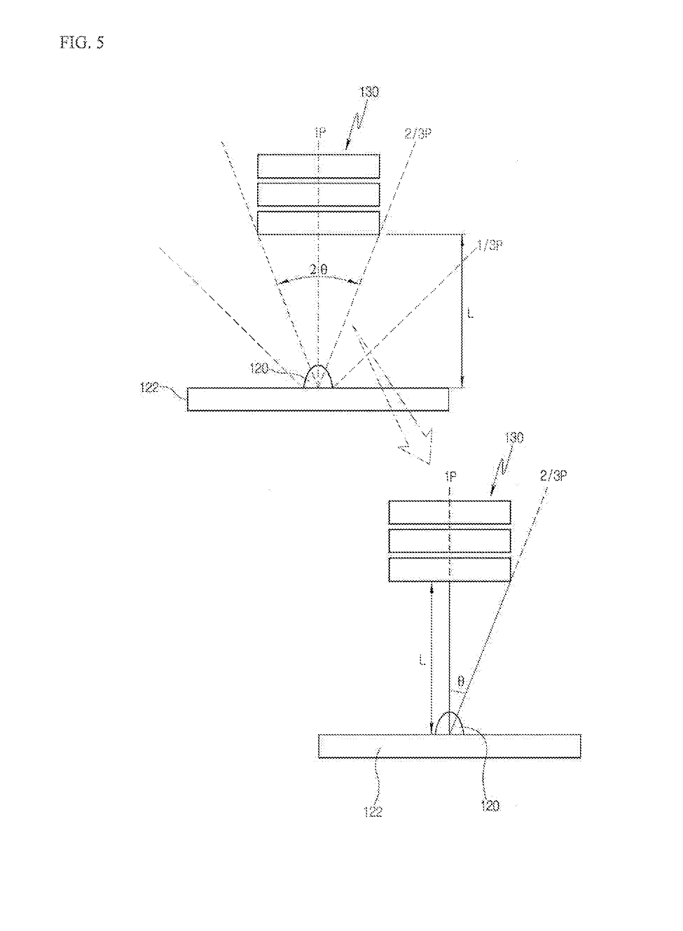

Hereinafter, a catalyst device and an air conditioning apparatus for a vehicle having the same in accordance with the present disclosure having the above-described characteristics will be described in detail with reference to the accompanying drawings. FIG. 2 is an exploded perspective diagram illustrating a catalyst device in accordance with a first embodiment of the present disclosure, FIG. 3 is a longitudinal cross-sectional diagram of the state in which FIG. 2 has been assembled, FIG. 4 is a diagram illustrating the spacing distance between the conventional light source part and catalyst part, and FIG. 5 is a diagram illustrating the spacing distance between the light source part and the catalyst part in accordance with the first embodiment of the present disclosure.

Referring to FIGS. 2 to 5, a catalyst device 100 in accordance with an embodiment of the present disclosure is configured to include a body 110, a light source part 120 for irradiating ultraviolet light, a catalyst part 130, a case 140, and a sealing part 150.

For example, the catalyst device 100 includes the case 140, the light source part 120 located to face the inside of the case 140 to irradiate light toward the inside surface of the case 140, and the catalyst part 130 located on the inside surface of the case 140 and generating the photocatalyst reaction by the light irradiated by the light source part 120, and the catalyst part 130 is spaced at a first spacing distance L apart from the light source part 120 so that the maximum optical energy Pmax of the light source part 120 is concentrated thereon.

Particularly, the catalyst part 130 in accordance with the present embodiment can easily concentrate the maximum optical energy Pmax of the light source part 120 thereon, thus stably deodorizing the evaporator regardless of the size reduction.

In addition, it is possible to locate the light source part 120 and the catalyst part 130 at the first spacing distance L to easily concentrate the maximum optical energy Pmax thereon, thus preventing the reduction in deodorization efficiency due to the size reduction.

The body 110 is mutually assembled with the case 140 to provide the space where the light source part 120 and the catalyst part 130, which will be described later, can be stably installed.

The body 110 has a receiving part 111 to seat a substrate 122, and the substrate 122 is provided with the light source part 120 for irradiating ultraviolet (UV) light having a first wavelength region on the upper surface thereof.

The case 140 can be configured such that the upper and lower cases are assembled with each other as illustrated in the figure, or can be separately composed of the left and right cases to be assembled with each other as well.

The light source part 120 in accordance with the present embodiment can selectively use any one of ultraviolet ray having a first wavelength region or visible ray having a second wavelength region.

Since the light source part 120 uses an LED, even when the evaporator is sterilized, it is harmless to the safety of the driver in the vehicle, and can be easily repaired even when a failure is caused. Accordingly, it is possible to simultaneously enhance the safety of passengers and the sterilization efficiency of the evaporator.

Since the socket 102 for power supply is provided on the substrate 122, it is possible to stably supply the power source when intending to supply it to the light source part 120. In addition, when the body 110 and the case 140 are assembled to each other on the element (not illustrated) provided on the substrate 122 or the light source part 120, it is possible to achieve the stable operation of the light source part 120.

Since the light source part 120 uses the LED, it is possible to safely use it because it does not cause any trouble to the passenger's health together with stable sterilization for the evaporator.

Before describing the light source part 120, the catalyst part 130 is extended with the same horizontal and vertical lengths, and for example, is extended with the length of the horizontal 44 mm*the vertical 44 mm.

When the horizontal length of the extended length of the catalyst part 130 is relatively shorter than the conventional used length and the ultraviolet light is irradiated to the catalyst part 130, the radiation is performed as the state that the optical amount of the optical energy has been changed.

That is, for the ultraviolet light irradiated from the light source part 120, the location, in which the maximum optical energy Pmax is concentrated due to the change in the spacing distance between the light source part 120 and the catalyst part 130, is changed, and it will be described in more detail with reference to the drawings.

FIG. 4 is a diagram illustrating the spacing distance between the light source part and the catalyst part in the related art, and FIG. 5 is a diagram illustrating the spacing distance between a light source part and a catalyst part in accordance with an embodiment of the present disclosure.

Referring to FIGS. 4 and 5, the spacing distance L between the light source part 12 and the catalyst part 13 in the related art is relatively shorter than the first spacing distance L between the light source part 120 and the catalyst part 130 in accordance with the present disclosure.

The length of the conventional catalyst part 13 is also extended to be relatively longer than that of the present disclosure, and for example, the conventional spacing distance L is kept at the spacing distance of 5 mm.

In this case, when ultraviolet light is irradiated from the light source parts 12, 120 having the same specification, the maximum light energies Pmax irradiated to the catalyst parts 13, 130 become differ between the related art and the present disclosure.

The maximum optical energy Pmax of the catalyst part 13 is kept in the state spaced at the distance corresponding to ⅓*P intensity based on 5 mm that is the vertical distance between the light source part 12 and the catalyst part 13 in the related art.

On the contrary, the first spacing distance L between the catalyst part 130 and the light source part 120 in accordance with the present embodiment is spaced at the distance corresponding to ⅔θP intensity covering only the lower surface region of the catalyst part 130 based on the vertical distance between the light source part 120 and the catalyst part 130 when the optical amount P is irradiated from the light source part 120.

Herein, the definition of the lower region is a region where the optical amount is concentrated when viewing the catalyst part 130 from the side surface thereof, and the entire range where the actual optical amount P reaches corresponds to ⅓*P.

In the related art, the maximum optical energy Pmax of ultraviolet light irradiated toward the catalyst part 13 is obtained at ⅓*P of the light source part 12. In this case, the manufacturing cost increases due to the increase in the size of the catalyst part 13 and the deodorization effect of the evaporator is excellent, but the cost is increased.

The present embodiment reduces the size of the catalyst part 130, and separates the first spacing distance L between the light source part 120 and the catalyst part 130 at a specific distance in order to improve the above conventional problems.

Then, it is possible to maximally irradiate the maximum optical energy Pmax irradiated from the light source part 120, thus stably deodorizing the evaporator without reducing efficiency due to the size reduction of the catalyst part 130.

In addition, although the size of the catalyst part 130 has been reduced as compared with the conventional one, the optical energy of the light source part 120 can be more concentrated, thus not generating an unnecessary dead zone.

For example, the first spacing distance L in accordance with the present embodiment is kept at 15 mm with respect to the catalyst part 130 based on the light source part 120. For the first spacing distance L, when the vertical spacing distance with the light source part 120 is adjusted to satisfy ⅔*P corresponding to the maximum optical energy Pmax of the light source part 120, the maximum optical energy Pmax of the light source part 120 is concentrated at 15 mm length as described above.

In this case, even when the ultraviolet light of the light source part 120 is maximally concentrated on the catalyst part 130 and the area thereof is reduced, the efficiency of the catalyst part 130 is excellently kept.

The first spacing distance L in accordance with the present embodiment is kept at the length of the catalyst part*½*tan θ/2. The first spacing distance L has the correlation with the size of the case 140 and the maximum optical energy of the light source part 120, and the optimal distance can be calculated using the above equation and can be applied to actual products.

In the light source part 120 in accordance with the present embodiment, the diffusion angle (θ) of the light source part 120 is kept within a range of 20 degrees to 60 degrees based on the maximum optical energy. The diffusion angle is applied when the catalyst part 130 is located in the region of the dotted line by the above-described first spacing distance L, as illustrated in the figure.

In this case, the maximum optical energy can be irradiated within a range of the diffusion angle, and accordingly, the efficiency of the catalyst part 130 can be maximally kept. For reference, the light source part 120 is irradiated toward the catalyst part 130 at the entire diffusion angles of a range of about 120 degrees.

The light source part 120 in accordance with the present embodiment uses the LED and thus, it is possible to safely use it because harmful components are not discharged or there is no potential danger, such that it is possible to easily repair and replace it even when a failure is caused, thus enhancing the operator's workability.

The LED irradiates Ultra Violet-A (UVA) light or Ultra Violet-C (UVC) light having a wavelength of 400 nm or less, and it is possible to solve the problem of mercury use which has been a problem in the conventional mercury lamp and to effectively irradiate light with a small power.

Since the UVA is relatively inexpensive, it is advantageous in terms of the cost, and it is possible to effectively activate the photocatalyst reaction of the catalyst part 130. The UVC is relatively expensive, but can activate the photocatalyst reaction and simultaneously perform its own sterilization function, thus enhancing the sterilization efficiency.

For reference, the light source part 120 can be composed of one or a plurality of light sources, and is not limited to a specific number.

The first wavelength region in accordance with the present embodiment has the optical amount P kept in a range of 180 nm to 360 nm, and the irradiation is selectively performed toward the catalyst part 130 in a range of the above-described first wavelength region.

For example, for the first wavelength region, the optical amount P can be irradiated to the catalyst part 130 in any range of the above-described range, and in this case, the efficiency can be excellently kept, such that the above-described optical amount is irradiated toward the catalyst part 130.

The light source part 120 can be composed of any one of one or a plurality of LEDs, and in this case, a plurality of the same LEDs can be provided therein.

The light source part 120 can be configured to simultaneously irradiate the LED irradiating ultraviolet light and visible light. In this case, due to the different wavelengths, the optical energy is concentrated on the catalyst part 130, thus enhancing the efficiency.

The second wavelength region in accordance with the present embodiment has the optical amount P kept in a range of 380 nm to 760 nm, and the irradiation is selectively performed toward the catalyst part 130 in a range of the above-described second wavelength region.

In addition, the second wavelength region in accordance with the present embodiment can have the optical amount P kept in a range of 400 nm to 500 nm. In this case, the irradiation is selectively performed toward the catalyst part 130 in a range of the above-described second wavelength range.

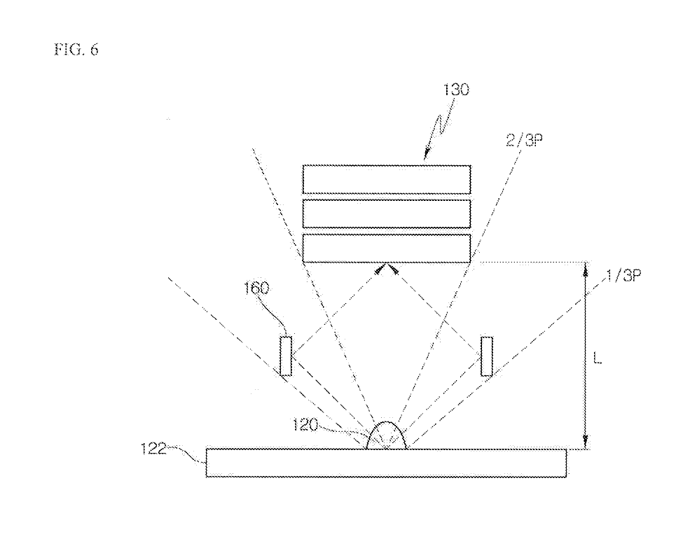

Referring to FIG. 6, the present embodiment includes a reflection plate 160 located to face the light source part 120 at the inside of the case 140 in order to reflect the light source irradiated from the light source part 120 to the catalyst part 130. The reflection plate 160 is located at the location between ⅔*P and ⅓*P based on the optical amount P.

The reflection plate 160 can be located to be perpendicular to the catalyst part 130 or can be located to be inclined at an angle of less than 5 degrees, and is not limited to the state illustrated in the figure.

The catalyst part 130 in accordance with the present embodiment generates the photocatalyst reaction by the light irradiated by the light source part 120 to generate peroxide radicals. The catalyst part 130 generates the photocatalyst reaction by the light irradiated from the light source part 120, and removes the contaminants introduced into the air conditioning case 300 due to the oxidizing action of the peroxide radicals generated in the photocatalyst reaction and fungus, various pollutants and odors in the evaporator 410.

When the catalyst part 130 absorbs the light irradiated from the light source part 120, the electrons of the Valence Band (VB), which is filled with electrons, absorb the light energy to move to the Conduction Band (CB) where the electrons are empty.

A hole that is an empty electron spot in the valence band oxidizes the water molecules on the surface, and the oxidized water molecule becomes OH radical.

In addition, the electrons called Excited Electrons excited by the conduction band react with oxygen to create the peroxide radicals having a strong acid power and sterilize the evaporator 410.

The catalyst part 130 in accordance with the present embodiment has the air porosity kept at 80% or more and the thickness of 5 mm to 50 mm.

The weight of the catalyst contained in the catalyst part 130 is kept within a range of 10% to 30% of the total weight of the catalyst part 130. The catalyst consists of titanium oxide particularized to a size of 10 nm to 60 nm.

The titanium oxide (TiO₂) receives ultraviolet ray of 400 nm or less to generate peroxide radicals, and the generated peroxide radicals decompose the organic substance into safe water and carbon dioxide.

The titanium oxide is a nano-encapsulated to generate a large amount of peroxide radicals even when using a light source having a relatively weak ultraviolet wavelength.

Accordingly, it has excellent decomposing ability of organic substance, has persistent durability and stability against environmental change, and has a semi-permanent effect. In addition, a large amount of the generated peroxide radicals can remove not only organic substance but also various substances such as odors and bacteria.

The catalyst part 130 forms a surface area value of nano-encapsulated titanium oxide at 330 m²/g or more, such that the number of particles that receive optical energy per the same area is much larger than that of general titanium oxide to increase the generated amount of the peroxide radicals.

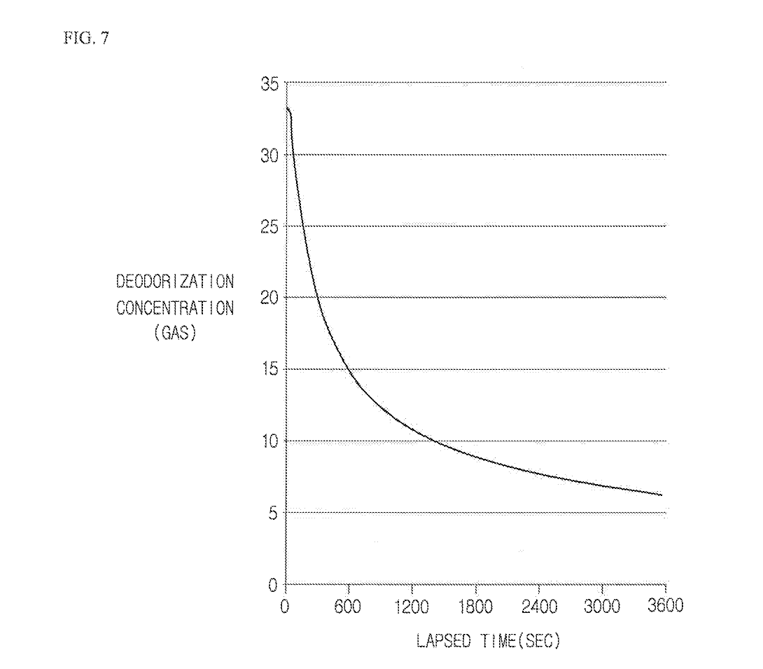

A graph illustrating the deodorization state depending upon the elapsed time through the catalyst device in accordance with the first embodiment of the present disclosure will be described with reference to FIG. 7. For reference, the X axis represents the elapsed time and the Y axis represents the deodorization concentration.

Referring to FIG. 7, when the catalyst part 130 in accordance with the present embodiment is spaced at the first spacing distance L apart from the light source part 120, the deodorization concentration depending upon the elapsed time is illustrated in the graph of the figure.

As described above, when the deodorization for the evaporator is performed through the catalyst part 130, the deodorization rate can be kept at 81.2% and the efficiency can be excellently kept even in the size reduction.

An air conditioning apparatus for a vehicle having a catalyst device in accordance with another embodiment of the present disclosure will be described with reference to the drawings.

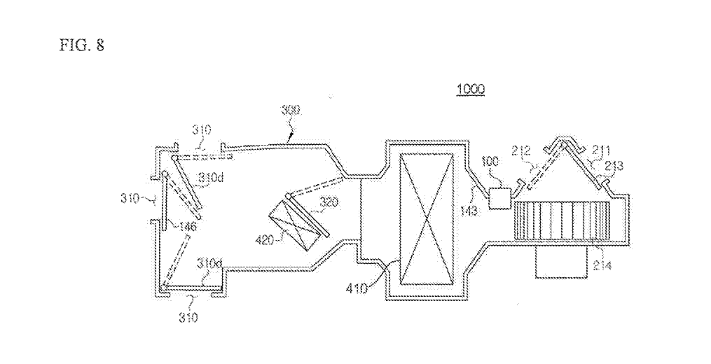

Referring to FIG. 8, an air conditioning apparatus for a vehicle 1000 includes the air conditioning case 300, the evaporator 410, a heater core 420, and the catalyst device 100.

The air conditioning case 300 transfers the inflow air therein, forms the space where the evaporator 410 and the heater core 420 are mounted therein, and forms a vent 310 through which the air is discharged therein.

More specifically, the air conditioning case 300 is formed with the vent 310 through which the temperature-controlled air is discharged by the evaporator 410 and the heater core 420 into the vehicle.

The vent 310 includes a Face Vent 310, a Defrost Vent 310, and a Floor Vent 310.

The Face Vent 310 discharges air to the front side (front seat) of the vehicle interior, the Defrost Vent 310 discharges air toward the windshield of the vehicle interior, and the Floor Vent 310 discharges air toward the bottom of the front seat of the vehicle interior, and the openings of the Face Vent 310, the Defrost Vent 310, and the Floor Vent 310 are adjusted through respective mode doors 310d.

A fan 214 for blowing air can be provided at the air inlet side of the air conditioning case 300, and an internal air inlet 211 and an external air inlet 212 can be selectively opened or closed by an internal/external air switching door 213, such that when the fan 214 is operated, the internal air or the external air is transferred to the air conditioning case 300.

The internal air inlet 211 communicates with the vehicle interior to inflow the internal air, and the external air inlet 212 communicates with the outside of the vehicle to inflow the external air.

The internal/external air switching door 213 is provided inside an inflow duct to open/close the internal air inlet 211 and the external air inlet 212, and the internal/external air switching door 213 operates depending upon the setting of the passengers in the vehicle to control so that the external air or the internal air is selectively flowed therein.

The evaporator 410 cools the air by the flow of the cold refrigerant, and the heater core 420 heats the air by the flow of the heated refrigerant, and the evaporator 410 and the heater core 420 are sequentially provided in the air flow direction.

In addition, the air conditioning case 300 is provided with a temperature control door 320 for determining the degree of the air from the evaporator 410 through the heater core 420 therein.

That is, the temperature control door 320 controls the opening of the warm air passage that all air passing through the evaporator 410 passes through the heater core 420, and the cool air passage that does not pass through the heater core 420.

In this time, the catalyst device 100 has the above-described characteristics, and can be provided at the front side of the evaporator 410 in the air flow direction to sterilize and deodorize the evaporator 410.

In addition, the catalyst device 100 is provided at the rear side of the evaporator 410 in the air flow direction to sterilize and deodorize the evaporator 410, and also, the peroxide radicals generated in the catalyst device 100 is flowed into the vehicle to perform air clean in the vehicle interior.

In addition, the air conditioning apparatus for the vehicle 1000 of the present disclosure is configured so that the catalyst device 100 is mounted at one side of the air conditioning case 300, and it is possible to easily mount and detach the catalyst device 100 and thereby to easily check and repair it, thus minimizing to disturb the air flow in the air conditioning case 300 the catalyst device 100.

The present disclosure is not limited to the above embodiments, and it will be apparent that the applicable range is various and various changes can be made without departing from the subject matter of the present disclosure as defined by the appended claims.

### INDUSTRIAL APPLICABILITY

The embodiments described in the present disclosure are applicable to an air conditioning apparatus provided in the vehicle that is an example of the transportation.

1. A catalyst device, comprising:
a case;
a light source part located to face the inside of the case to irradiate light toward the inside surface of the case; and
a catalyst part located on the inside surface of the case, and generating the photocatalyst reaction by the light irradiated by the light source part,
wherein the catalyst part is located to be spaced at a first spacing distance L apart from the light source part so that the maximum optical energy Pmax of the light source part is concentrated thereon.

2. The catalyst device of claim 1, wherein the light source part selectively uses any one of ultraviolet ray having a first wavelength region or visible ray having a second wavelength region.

3. The catalyst device of claim 1, wherein the first spacing distance L is kept in the state spaced at the distance corresponding to ⅔*P intensity covering only the lower surface region of the catalyst part based on the vertical distance between the light source part and the catalyst part when the optical amount P is irradiated from the light source part.

4. The catalyst device of claim 1, wherein the first spacing distance L is kept at the length of the catalyst part*½*tan θ/2.

5. The catalyst device of claim 1, wherein the first spacing distance L with the catalyst part based on the light source part is kept at 15 mm.

6. The catalyst device of claim 1, wherein the diffusion angle (θ) of the light source part based on the maximum optical energy of the light source part is in a range of 20 degrees to 60 degrees.

7. The catalyst device of claim 1, wherein the light source part uses an LED.

8. The catalyst device of claim 1, wherein the light source part is composed of any one of one or in plural.

9. The catalyst device of claim 1, wherein the catalyst part is extended with the same horizontal and vertical lengths.

10. The catalyst device of claim 2, wherein the first wavelength region has the optical amount P kept in a range of 180 nm to 380 nm.

11. The catalyst device of claim 2, wherein the second wavelength region has the optical amount P kept in a range of 380 nm to 760 nm.

12. The catalyst device of claim 2, wherein the second wavelength region has the optical amount P kept in a range of 400 nm to 500 nm.

13. The catalyst device of claim 1, comprising a reflection plate located to face the light source part on the inside of the case to reflect the light source irradiated from the light source part to the catalyst part.

14. The catalyst device of claim 1, wherein the reflection plate is located at the location between ⅔*P and ⅓*P based on the optical amount P.

15. The catalyst device of claim 1, wherein the catalyst part has the air porosity kept at 80% or more.

16. An air conditioning apparatus for a vehicle, comprising:
an air conditioning case for forming a space where inflow air is transferred to form a vent through which the air is discharged;
an evaporator provided inside the air conditioning case;
a heater core provided at the rear side of the air conditioning case in the air flow direction; and
a catalyst device 100 of according to claim 1.

17. The air conditioning apparatus for the vehicle of claim 16, wherein the catalyst device is provided at the front side of the evaporator in the air flow direction.

18. The air conditioning apparatus for the vehicle of claim 16, wherein the catalyst device is provided at the rear side of the evaporator in the air flow direction.
